(19) United States
(12) Patent Application Publication
(10) Pub. No.: US 2023/0131120 A1
(43) Pub. Date: Apr. 27, 2023

(54) METHODS FOR BODY WEIGHT CONTROL USING THIOLATED CHITOSANS

(71) Applicant: National Health Research Institutes, Zhunan Town (TW)

(72) Inventors: Feng-Huei LIN, Miaoli County (TW); Ching-Yun Chen, Miaoli County (TW)

(73) Assignee: National Health Research Institutes, Zhunan Town (TW)

(21) Appl. No.: 17/971,753

(22) Filed: Oct. 24, 2022

(30) Foreign Application Priority Data
Oct. 25, 2021 (TW) 110139574

(51) Int. Cl. A61K 31/722; A61P 3/04; A61K 9/00

(52) U.S. Cl. CPC A61K 31/722; A61P 3/04; A61K 9/0053

(57) ABSTRACT

The present invention relates to a method for body weight control in a subject in need thereof, comprising administering an effective amount of a thiolated chitosan to the subject. In particular, the thiolated chitosan of the present invention comprises a plurality of thiol groups covalently bonded to a chitosan moiety thereof. The thiolated chitosan of the present invention can be orally administered, which is effective in slowing down body weight gain and has good biocompatibility.

### RELATED APPLICATIONS

This application claims the benefit of Taiwan Patent Application No. TW110139574, filed on Oct. 25, 2021, the entire content of which is incorporated herein by reference.

### TECHNOLOGY FIELD

The present invention relates to a method for body weight control in a subject in need thereof, comprising administering an effective amount of a thiolated chitosan to the subject. In particular, the thiolated chitosan of the present invention comprises a plurality of thiol groups covalently bonded to a chitosan moiety thereof. The thiolated chitosan of the present invention can be orally administered, which is effective in slowing down body weight gain and has good biocompatibility.

### BACKGROUND OF THE INVENTION

With the advancement of industry and the development of science and technology, the situation of insufficient food in various countries has gradually become food surplus. The problem of undernourishment had been solved, but the problem of obesity has emerged (Nature 404(6778) (2000) 635). Obesity has many negative effects on health. For example, it increases the risk of hypertension, coronary artery disease and stroke (Journal of human hypertension 11(11) (1997); International journal of obesity 28(11) (2004) 1357-1364), and is also highly correlated with type 2 diabetes. Obesity is already a health problem that must be paid attention to worldwide.

Diet control and exercise are the most common ways of body weight control, which increase calorie consumption, maintain a normal weight and avoid obesity by avoiding excessive intake of calories and increasing exercise. The U.S. Food and Drug Administration (FDA) has approved a variety of weight loss drugs that must be used a physician's evaluation. For example, Roche Xenical (Orlistat, Xenical®) can inhibit the absorption of fat in the small intestine, but the side effects are oily stools, increased excretion frequency and deficiency of fat-soluble vitamins. In addition, a variety of weight management procedures have been developed, including gastric band surgery, gastric bypass surgery, gastric sleeve resection, intragastric water polo and so on. Patients continue to cooperate with the normal course of treatment after surgery and almost all have obvious results, but surgery has its risks and may cause sequelae (Obesity surgery 12(5) (2002) 652-660; New England Journal of Medicine 357(8) (2007) 753-761; Obesity surgery 17(10) (2007) 1297; The American journal of clinical nutrition 48(3) (1988) 592-594). The duodenal lining (EndoBarrier) is a recently developed medical device, which is delivered by a gastroscope in an invasive manner to the stomach and the middle of duodenum, where a plastic membrane is provided to block the intestinal absorption of nutrients to achieve the purpose of body weight control (Surgery for Obesity and Related Diseases 9 (3) (2013) 482-484). However, in addition to the pain and risk caused by invasive delivery to the patient, gastrointestinal peristalsis may cause the device to fall off, the device may cause damages of abnormal proliferation to the surrounding tissues, and long-term blocking of nutrient absorption causes harm to the patient's health; thus it must be taken out within a certain period of time. In addition, if the weight loss approach is too intense and causes the weight to drop too quickly, it may cause harm to the body, such as malnutrition, anemia, osteoporosis, endocrine disorders, etc.

There is still a need in this field to develop simple, convenient and low-risk technical means to achieve effective body weight control and avoid obesity.

### SUMMARY OF THE INVENTION

In one aspect, the present invention provides a method for body weight control, comprising administering an effective amount of a thiolated chitosan to the subject.

In some embodiments, the thiolated chitosan comprises a plurality of thiol groups covalently bonded to a chitosan moiety thereof.

In some embodiments, the thiolated chitosan is partially thiolated.

In some embodiments, the thiolated chitosan has a degree of thiolation of 0.5% to 50%.

In some embodiments, the thiolated chitosan has a degree of thiolation of 30 to 5,000 μmol/g.

In some embodiments, the thiolated chitosan has an average molecular weight of 5 to 2,000 kDa.

In some embodiments, the thiolated chitosan is represented by formula I:

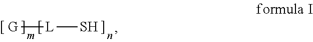

wherein

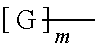

represents a chitosan moiety, G is a monomer unit of the chitosan moiety, including N-acetyl-D-glucosamine monomer and glucosamine monomer, in which the monomer units are connected to form the chitosan moiety, and m represents a total number of the monomers, being an integer from 30 to 12,500;

SH represents a thiol group, and L represents a linker moiety; and

n is an integer from 1 to 6,250 and n is not more than m.

In some embodiments, L is a bond, or a molecular moiety including one or more functional groups containing heteroatoms, which is selected from the group consisting of amido, amino, carbonyl, carbamate, urea, disulphide, ether, succinyl, succinamidyl and a combination thereof.

In some embodiments, L is —R—C(O)—R′—, where R and R′ are each independently a bond, or R and R′ are each independently a C1-20 alkylene group, optionally containing 1 to 3 oxygen atoms in its main chain, unsubstituted or substituted with 1 to 3 hydroxyl groups (—OH).

In some embodiments, L is —R—C(O)—R′—, where R is a C1-6 alkylene group, and R′ is a C1-18 alkylene group containing 1 to 3 oxygen atoms in its main chain and substituted with 1 to 3 hydroxyl groups (—OH).

In some embodiments, L is represented by formula Ia

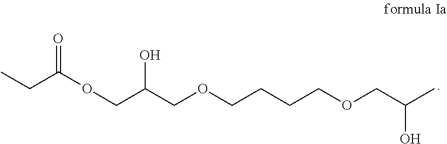 [SMILES: CCC(=O)OCC(O)COCCCCOCC(C)O]

In some embodiments, m is an integer from 30 to 300, an integer from 300 to 1,000, an integer from 1,000 to 2,500, or an integer from 2,500 to 10,000.

In some embodiments, the thiolated chitosan has an average molecular weight of 5 to 50 kDa, 50 to 200 kDa, 200 to 500 kDa, or 500 to 2,000 kDa.

In some embodiments, the thiolated chitosan is administered in an amount effective in slowing down or inhibiting weight gain.

In some embodiments, the thiolated chitosan is administered in an amount effective in treating or avoiding overweight or obesity.

In some embodiments, the thiolated chitosan is formulated with a physiologically acceptable carrier to form a composition.

In some embodiments, the composition is in a form of food or medicine.

In some embodiments, the composition is in a form of tablets, capsules, powder, emulsions and aqueous suspensions.

In some embodiments, the thiolated chitosan or a composition thereof is administered orally.

In some embodiments, the thiolated chitosan or a composition thereof is administered daily or every other day for a period of 1 to 6 weeks or longer.

In another aspect, the present invention provides a method for body weight control, which comprises administering the thiolated chitosan described herein to a subject in need. The present invention also provides a composition for body weight control, which comprises the thiolated chitosan described herein and optionally a physiologically acceptable carrier.

The details of one or more embodiments of the present invention are set forth in the following description. Other features or advantages of the present invention will become apparent from the following drawings and the detailed description of several specific embodiments, and also from the appended claims.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing summary, as well as the following detailed description of the invention, will be better understood when read in conjunction with the appended drawings. For the purpose of illustrating the invention, there are shown in the drawings the embodiments which are presently preferred. It should be understood, however, that the invention is not limited to the precise arrangements and instrumentalities shown.

In the drawings:

FIG. 1 shows the infrared spectroscopy analysis results of the thiolated chitosan sample and the unmodified chitosan sample.

FIG. 2 shows the nuclear magnetic resonance spectroscopy analysis results of the unmodified chitosan sample.

FIG. 3 shows the nuclear magnetic resonance spectroscopy analysis results of the thiolated chitosan sample.

FIG. 4 shows the cytotoxicity analysis results (WST-1 assay) of the thiolated chitosan sample and the unmodified chitosan sample.

FIG. 5 shows the cytotoxicity analysis results (live/dead assay) of the thiolated chitosan sample and the unmodified chitosan sample.

FIG. 6 shows the results of the attachment ability analysis of the thiolated chitosan sample and the unmodified chitosan sample using the in vitro gastrointestinal tract simulation model.

FIG. 7 shows the daily body weight of animals in each group, including: (1) Control, (2) 1×unmodified Chitosan, (3) 1×thiolated Chitosan (1×Chirosan-TGA), and (4) 0.5×thiolated Chitosan (0.5×Chirosan-TGA).

FIG. 8 shows the weekly body weight percentages of the animals in three groups, including 1×unmodified Chitosan, 1×thiolated Chitosan (1×Chirosan-TGA), and 0.5×thiolated Chitosan (0.5×Chirosan-TGA), which were calculated based on the body weight of the animals in the control group as 100% of the current week.

FIG. 9 shows the results of organ weight analysis of animals in each group, including: (1) Control, (2) 1×unmodified Chitosan, (3) 1×thiolated Chitosan (1×Chirosan-TGA), and (4) 0.5×thiolated Chitosan (0.5×Chirosan-TGA).

FIG. 10 shows the photos of the appearance of the organs of animals in each group, including: (1) Control, (2) 1×unmodified Chitosan, (3) 1×thiolated Chitosan (1×Chirosan-TGA), and (4) 0.5×thiolated Chitosan (0.5×Chirosan-TGA).

### DETAILED DESCRIPTION OF THE EMBODIMENTS

Unless defined otherwise, all technical and scientific terms used herein have the same meaning as commonly understood by a person skilled in the art to which this invention belongs. As used herein, the articles “a” and “an” refer to one or more than one (i.e., at least one) of the grammatical object of the article. By way of example, “an element” means one element or more than one element. As used herein, the term “comprise” or “comprising” is generally used in the sense of include/including which means permitting the presence of one or more features, ingredients or components. The term “comprise” or “comprising” encompasses the term “consists” or “consisting of” As used herein, the term “about” or “approximately” refers to a degree of acceptable deviation that will be understood by persons of ordinary skill in the art, which may vary to some extent depending on the context in which it is used. In general, “about” or “approximately” may mean a numeric value having a range of ±10%, ±5% or ±3% around the cited value

As described herein, the term “chitin” refers to a carbohydrate polymer mainly composed of N-acetyl-glucosamine monomer units, which exists in many biological sources, for example, shells of crustaceans (shrimp, crabs), exoskeletons of insects (locusts, scarabs) and cartilage of molluscs (squid, shellfish).

As described herein, the term “chitosan” refers to a product obtained by deacetylation of chitin, which may include acetylglucosamine monomer units (the non-deacetylated portion) and glucosamine monomer units (the deacetylated portion) in structure, in which the number of glucosamine monomer units (the deacetylated portion) divided by the total number of monomer units of the non-deacetylated portion and the deacetylated portion indicates a degree of deacetylation of chitosan (Reactive and functional polymers 46(1) (2000) 1-27). In some embodiments, the degree of deacetylation of chitosan described herein is greater than 50%, for example, 55% to 99%, generally 60% to 95% or more, including 60%, 65%, 70%, 75%, 80%, 85%, 90%, 95% or more. In certain embodiments, the degree of deacetylation of chitosan described herein is 90% to 95% or more. The deacetylation of chitin can be carried out in a manner known in the art, for example, treatment with high temperature and strong alkali. Chitosan has a pKa value of approximately 6.5, and when it is dissolved in the acidic environment, the amino group (NH₂) in the structure is exposed and protonated to NH³, making it positively charged in the solution and easy to polymerize with negatively charged molecules, thereby having a wide range of applicability and easy modification characteristics conferred by the amino group (Journal of Polymer Research 11(2) (2004) 141-147; Carbohydrate Polymers 78(4) (2009) 773-778). The higher the degree of polymerization of chitosan, the higher the molecular weight, the higher the viscosity, and the absorption is strong, while the lower the degree of polymerization, the smaller the molecular weight, the lower the viscosity, the better the water solubility, and the easier the absorption by the body.

As described herein, the term “thiolated chitosan” refers to a chitosan modified (or reformed or grafted) with thiol(s). Specifically, a thiolated chitosan has a chitosan moiety (main chain) attached with a side chain with a thiol group.

As described herein, the term “aliphatic” or “aliphatic group” represents a hydrocarbon moiety, which may be straight, branched or cyclic, and may be completely saturated or may contain one or more units of unsaturation. In general, an aliphatic group contains 1-30 carbon atoms. In some embodiments, an aliphatic group contains 1-20 carbon atoms, for example, 1-12 carbon atoms, 1-8 carbon atoms or 1-6 carbon atoms. In some embodiments, an aliphatic group contains 2-20 carbon atoms, for example, 2-12 carbon atoms, 2-8 carbon atoms or 2-6 carbon atoms. In some embodiments, an aliphatic group contains 3-20 carbon atoms, for example, 3-12 carbon atoms, 3-8 carbon atoms or 3-6 carbon atoms. In some embodiments, an aliphatic group contains 4-20 carbon atoms, for example, 4-12 carbon atoms, 4-8 carbon atoms or 4-6 carbon atoms. Suitable aliphatic groups include, but are not limited to, straight or branched alkyl, alkylene, alkenyl, alkenylene, alkynyl, alkynylene, aryl, arylene and a hybrid thereof, for example, (cycloalkyl)alkyl, (cycloalkenyl)alkyl, aralkylene, or (cycloalkyl)alkenyl.

As used herein, the term “alkyl” refers to a straight or branched monovalent hydrocarbon containing 1-30 carbon atoms. Examples of alkyl groups include, but are not limited to, methyl, ethyl, n-propyl, isopropyl, n-butyl, isobutyl, and tertiary butyl. The term “alkenyl” refers to a straight or branched monovalent hydrocarbon containing 2-30 carbon atoms and one or more double bonds. Examples of alkenyl include, but are not limited to, ethenyl, propenyl, and 1- and 2-butenyl. The term “alkynyl” refers to a straight or branched monovalent hydrocarbon containing 2-30 carbon atoms and one or more triple bonds. Examples of alkynyl groups include, but are not limited to, ethynyl, 1-propynyl, and 1- and 2-butynyl. The term “aryl” refers to a monovalent 6-carbon monocyclic, 10-carbon bicyclic, 14-carbon tricyclic aromatic ring system. Examples of aryl groups include, but are not limited to, phenyl, naphthyl, and anthracenyl. The term “heteroaryl” refers to a monovalent aromatic 5-8 membered monocyclic ring, 8-12 membered bicyclic ring, or 11-14 membered tricyclic ring system with one or more heteroatoms (for example, O, N, S, or Se). Examples of heteroaryl groups include pyridyl, furyl, imidazolyl, benzimidazolyl, pyrimidinyl, thienyl, quinolinyl, indolyl, and thiazolyl. As used herein, “alkylene”, “alkenylene”, “alkynylene”, “arylene” and “heteroarylene” refer to a divalent group of alkyl, alkenyl, alkynyl, aryl and heteroaryl. Examples of alkylene include, but are not limited to, methylene and ethylene. Examples of alkenylene include, but are not limited to, vinylene (—CH═CH—) and propenylene (—CH═CHCH₂—, —CH₂—CH═CH—). Examples of alkynylene include, but are not limited to, ethynylene, propynylene, and the like. Examples of arylene include, but are not limited to, phenylene (—(C₆H₄)—), such as m-phenylene and p-phenylene; and examples of heteroarylene include, but are not limited to, pyridine (—(C₅H₃N)—) and furan (—(C₄H₂O)—).

The aliphatic groups described herein, for example, alkyl, alkylene, alkenyl, alkenylene, alkynyl, alkynylene, aryl, arylene, heteroaryl and heteroalkylene groups, include substituted and unsubstituted moieties. Examples of substituents include, but are not limited to, hydroxyl, amine, halogen (for example, F, Cl, Br and I), alkyl, alkenyl, alkynyl, alkoxy, aryl and heteroaryl.

In the present invention, it is found that thiolated chitosan has the effects of inhibiting body weight gain, which is better than unmodified chitosan. Therefore, the present invention provides use of a thiolated chitosan for manufacturing a composition for body weight control. The present invention also provides a method for body weight control, which comprises administering a thiolated chitosan described herein to a subject in need. The present invention also provides a composition for body weight control, which includes a thiolated chitosan described herein and optionally a physiologically acceptable carrier.

The small intestine has four layers in structure, including mucosa, sub-mucosa, muscularis externa and serosa, with villi and microvilli specialized on the surface to increase the efficiency of nutrient absorption. In addition, the small intestine secretes a layer of mucosa in the surface, which can protect the surface of the intestinal tract from physical and chemical damages. The main components of the mucosa are water, mucin, lipids and salts, in which mucin confers adhesion to the mucosa (Cellular and molecular life sciences 68(22) (2011) 3635). Mucin is a glycoprotein, which has a main backbone of PTS structure composed of abundant proline, threonine and serine, and is modified by many sugar groups on the side chain to form a structure similar to a bottle brush, with globular protein and a cysteine-rich domain (Current opinion in colloid & interface science 11(2) (2006) 164-170). Common redox system members in organisms include glutathione (GSH), cysteine (Cys) and thioredoxin (Trx), which play an important physiological regulation function in organisms and can maintain redox stability, metabolic function and cell integrity of tissues. In the intestinal environment, the reduction reaction can maintain mucus, absorb nutrients and prevent damage caused by free radicals.

According to the present invention, a thiolated chitosan is mucoadhesive and can be used as a mucoadhesive material. After it enters the front of the small intestine, it may be attached to the intestinal mucosa to form a film, which blocks direct contact between food and the intestinal tract, thereby reducing nutrient absorption and achieving the effect of body weight control. In the intestinal environment, the thiolated chitosan may form a covalent disulfide bond with the cysteine residues of mucin on the intestinal mucosa through the thiol group on its side chain by oxidation reaction and produces adhesion with intestinal mucosa, which can exhibit higher adhesion than mucosal adhesion materials that rely on hydrogen bonds and Van der Waals force. On the other hand, the reduction reaction of the intestinal environment may break part of the covalent disulfide bonds formed between the thiolated chitosan and the mucosa, so that the thiolated chitosan will not be permanently fixed on the mucosa and will not cause damages to the intestine or block nutrient absorption for a long time. In general, the higher the degree of thiolation of the thiolated chitosan, the more the covalent disulfide bonds that may be formed with the mucosa, and the higher the adhesion to the intestinal mucosa. Conversely, the lower the degree of thiolation of thiolated chitosan, the fewer the covalent disulfide bonds that may be formed with the mucosa, and the lower the adhesion to the intestinal mucosa. The thiolation of the thiolated chitosan can be adjusted as needed to achieve the desired adhesion.

The “degree of thiolation” with respect to a thiolated polymer described herein refers to the content of thiol groups on the polymer. The degree of thiolation of the thiolated polymer can be determined by methods known in the art. For example, Ellman's assay can quantify the thiol content of a thiolated chitosan (expressed in μmol SH/g). In addition, Ninhydrin assay can measure the number of amino groups of chitosan before modification and that of a thiolated chitosan after modification, which can be entered into the following formula to calculate the modification rate of the thiolated chitosan after modification:

$$\frac{\begin{matrix}the number of amino groups of chitosan before modification - \\ the number of amino groups of a \\ thiolated chitosan after modification\end{matrix}}{the number of amino groups of chitosan before modification} \times 100 \%$$

In some embodiments, the thiolated chitosan of the present invention has a degree of thiolation of 0.5% to 50%. In some examples, the thiolated chitosan of the present invention has a degree of thiolation of 0.5% to 40%, 0.5% to 30%, 0.5% to 20% or 0.5% to 10%.

In some embodiments, the thiolated chitosan of the present invention has a degree of thiolation of 30 to 5,000 μmol/g. In some embodiments, the thiolated chitosan of the present invention has a degree of thiolation of 30 to 2,500 μmol/g, 30 to 1,500 μmol/g, 30 to 700 μmol/g, 30 to 500 μmol/g or 30 to 250 μmol/g.

The modified chitosan of the present invention may have an average molecular weight in various ranges, for example, an average molecular weight of about 5 to 2,000 kDa. In some embodiments, the modified chitosan of the present invention has an ultra-low average molecular weight, for example, less than about 50 kDa (e.g. 5 to 50 kDa). In some embodiments, the modified chitosan of the present invention has a low molecular weight, for example, about 50 to about 200 kDa. In some specific embodiments, the modified chitosan of the present invention has a medium molecular weight, for example, about 200 to about 500 kDa. In some specific embodiments, the modified chitosan of the present invention has a high molecular weight, for example, about 500 kDa or more (e.g. 500 to 2,000 kDa).

In some embodiments, the thiolated chitosan of the present invention is represented by formula I:

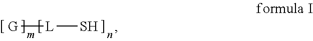

wherein

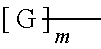

represents a chitosan moiety, G is a monomer unit of the chitosan moiety, including N-acetyl-D-glucosamine monomer and glucosamine monomer, in which the monomer units connected to form the chitosan moiety, and m represents a total number of monomers, being an integer from 30 to 12,500;

SH represents a thiol group, and L represents a linker moiety; and

n is an integer from 1 to 6,250 and n is not more than m.

In some embodiments, the linker moiety L of formula I is connected to the amino group (—NH₂) of the glucosamine monomer of the chitosan moiety and is connected to SH.

In some embodiments, the linker moiety L of Formula I may be a bond, or a molecule moiety including one or more functional groups containing heteroatoms, including but not limited to: amido (—C(O)NH—), amino (—NR—, R may be hydrogen or an aliphatic hydrocarbon group), carbonyl (—C(O)—), carbamate (—NHC(O)O—), urea (—NHC(O)NH—), disulfide (—S—S—), ether (—O—), succinyl (—(O)CCH₂CH₂C(O)—), succinamidyl (—NHC(O)CH₂CH₂C(O)NH—) and any combination thereof. For example, the linker may simultaneously include a carbamate molecule moiety (—NHC(O)O—) and an amido molecule moiety (—C(O)NH—).

In some embodiments, the linker moiety L of formula I is a C1-20 alkylene group, optionally containing 1 to 3 oxygen atoms in the main chain, unsubstituted or substituted with 1 to 3 hydroxyl groups (—OH).

In some embodiments, the linker moiety L of formula I is —R—C(O)—R′—, wherein R and R′ are each independently a bond, or R and R′ are each independently a C1-20 alkylene group, optionally containing 1 to 3 oxygen atoms in the main chain, unsubstituted or substituted with 1 to 3 hydroxyl groups (—OH).

L is -R-C(0)-R′-, where R is a C1-6 alkylene group, and R′ is a C1-18 alkylene group containing 1 to 3 oxygen atoms in its main chain and substituted with 1 to 3 hydroxyl groups (—OH)

L is represented by formula Ia

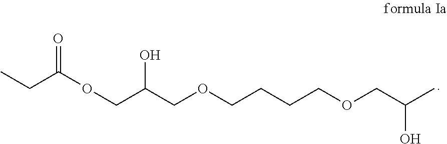 [SMILES: CCC(=O)OCC(O)COCCCCOCC(C)O]

In some embodiments, m is an integer from 30 to 300, an integer from 300 to 1,000, an integer from 1,000 to 2,500, or an integer from 2,500 to 10,000.

The modified chitosan of the present invention can be prepared in a manner known in the art. Typically, a cross-linking agent can be used to perform cross-linking reaction between chitosan and a molecule with a thiol group so as to connect the thiol group and the chitosan to each other, thereby forming a thiolated chitosan.

In some embodiments, examples of the cross-linking agent include, but are not limited to: a carbodiimide cross-linker, a biscarbodiimide cross-linker, a divinyl sulfone cross-linker, a diepoxy cross-linker, a disulfide cross-linker, a diglycidyl cross-linker, a diacrylate cross-linker and any combinations thereof.

In some embodiments, the cross-linking reaction for preparing a modified chitosan is carried out by using a butanediol diglycidyl ether (BDDE) cross-linker. The epoxy groups at both ends of the BDDE crosslinker will bond with the amino group (—NH₂) on the chitosan structure, and the BDDE crosslinker itself will be decomposed into 1,4-butanediol and glycerol after being hydrolyzed. Among them, 1,4-butanediol can be quickly oxidized to carbon dioxide by organisms, and glycerol can also be decomposed in the biological environment, which is not harmful to organisms and is safe.

For the purpose of delivery and absorption, an effective amount of the active ingredient can be formulated with a physiologically acceptable carrier to form a composition in an appropriate form. Depending on the mode of administration, the composition of the present invention may contain about 0.1% to about 100% by weight of the active ingredient, wherein the percentage is calculated based on the total weight of the composition.

The term “effective amount” as used herein refers to the amount of the active ingredient that imparts the desired biological effect in the individual or cell to be treated. The effective amount may vary depending on various reasons, such as the route and frequency of administration, the weight and species of the individual receiving the active ingredient, and the purpose of administration. Based on the content disclosed herein, the methods established, and their own experience, those skilled in the art can determine the dosage in each case.

The term “physiologically acceptable” as used herein means that the carrier is compatible with the active ingredient of the composition, and preferably can stabilize the active ingredient and is safe for the individual receiving it. The carrier can be a diluent, carrier, excipient, or matrix for the active ingredient. Some examples of suitable excipients include lactose, glucose, sucrose, sorbitol, mannitol, starch, Arabic gum, calcium phosphate, alginate, tragacanth gum, gelatin, calcium silicate, microcrystalline cellulose, polyvinylpyrrolidone, cellulose, sterile water, syrup and methylcellulose. The composition may additionally contain lubricants, such as talc, magnesium stearate and mineral oil; wetting agents; emulsifiers and suspending agents; preservatives, such as methyl and propyl hydroxybenzoates; sweeteners; and flavoring agents. After being administered to a patient, the composition of the present invention can provide the effect of rapid, continued or delayed release of the active ingredient.

The composition of the present invention is preferably administered orally. Orally acceptable dosage forms include, but are not limited to, tablets, capsules, powder, emulsions and aqueous suspensions. In certain embodiments, commonly used carriers for tablets include lactose and corn starch. Generally, a lubricant such as magnesium stearate can also be added to the tablets. When necessary, some sweeteners, flavors or coloring agents can be added.

As used herein, the term “prevention” refers to preventive measures against diseases or symptoms or conditions of diseases. Preventive measures include, but are not limited to, administering one or more active agents to individuals who have not been diagnosed with the disease or the symptoms or conditions of the disease but may be susceptible to or tend to suffer from the disease. The purpose of preventive measures is to avoid, prevent, or delay the occurrence of the disease or symptoms or conditions of the disease.

As used herein, the term “treatment” refers to therapeutic measures to a disease or the symptoms or conditions of a disease, which include but are not limited to applying or administering one or more active agents to a subject suffering from the disease or the symptoms or conditions of the disease or exacerbation of the disease. The purpose of the therapeutic measures is to treat, cure, mitigate, relieve, alter, remedy, ameliorate, improve, or affect the disease, the symptoms or conditions of the disease, disability caused by the disease, or exacerbation of the disease.

As used herein, a subject in need according to the present invention includes humans and non-human mammals. Non-human mammals include, but are not limited to, companion animals such as cats, dogs and the like, and farm animals such as cows, horses, sheep, goats, pigs and the like.

In certain embodiments, the composition of the present invention can be used as food or medicine.

According to the present invention, the thiolated chitosan as an active ingredient can be used for body weight control. Specifically, the thiolated chitosan of the present invention, as an active ingredient, is administered in an amount effective in slowing down the weight gain of an individual and can be used for weight loss. In some embodiments, the thiolated chitosan of the present invention can reduce the body weight of the individual who takes it daily or every other day for a period of 1 to 6 weeks by about 1% to about 10%, compared with the reference body weight of the individual who is not administered with the thiolated chitosan. Optionally, the dosage of the thiolated chitosan can be adjusted to achieve the desired weight loss rate. For example, the thiolated chitosan can be adjusted to be administered in a higher dose to achieve a relatively large reduction in the body weight of the individual; for example, taking the thiolated chitosan daily or every other day in an amount for a period of 1 to 6 weeks or longer (e.g. 8 weeks, 12 weeks, 24 weeks or more) to reduce the body weight by about 3% to about 10% (compared to the reference body weight of the individual who is not administered with the thiolated chitosan), while the thiolated chitosan can be adjusted to a lower dose to achieve a relatively small reduction in the body weight of the individual; for example, taking the thiolated chitosan daily or every other day in an amount for a period of 1 to 6 weeks (e.g. 8 weeks, 12 weeks, 24 weeks or more) to reduce the body weight by about 1% to about 5% (compared to the reference body weight without administration of the thiolated chitosan).

The World Health Organization uses the Body Mass Index (BMI) to measure the degree of obesity. The calculation formula is weight (kg) divided by the square of height (meters). According to the definition of the World Health Organization, normal individuals have a BMI value of 18.5 to 24.9, a BMI value greater than 25.0 is considered overweight, and a BMI value greater than 30.0 is considered obese. Among them, a BMI value of 30-34.9 is the first degree of obesity, and a BMI value of 35-39.9 is the second degree of obesity, and a BMI value of 40 or more is the third degree of obesity.

In some embodiments, the thiolated chitosan of the present invention is administered to normal individuals for preventing overweight or obesity.

In some embodiments, the thiolated chitosan of the present invention is administered to individuals who are overweight or obese. By slowing down or inhibiting weight gain, the desired therapeutic effect (for example, lowering BMI to arrive at a normal value) can be achieved.

The thiolated chitosan of the present invention is biologically compatible, safe, non-toxic, and degradable. After being taken orally and entering into the front of the small intestine, the thiolated chitosan is expected to adhere to the intestinal mucosa to form a film barrier, which avoids direct contact between food and the intestinal mucosa, thereby reducing absorption and achieving the effect of body weight control. This approach avoids invasive delivery, and the material is safe, non-toxic and degradable. It does not require a second operation to remove the device out, and provides a simple, convenient, low-risk and gentle and effective method to achieve the purpose of body weight control or treatment of overweight or obesity.

The present invention is further illustrated through the following examples, which are provided for illustration rather than limitation. Based on the content of the present invention, those skilled in the art should understand that without departing from the spirit and scope of the present invention, many changes can be made to the specific embodiments disclosed and still obtain the same or similar results.

### EXAMPLES

### Example 1

### Preparation of the Thiolated Chitosan

1 g of chitosan was dissolved in 50 mL of 0.1 M acetic acid solution, and 0.37 mL of thioglycolic acid (TGA) was added and stirred evenly. 0.098 mL of 1,4-butanediol diglycidyl ether (BDDE) was dissolved in 50 ml of isopropanol. The two solutions were mixed and stirred at room temperature and protected from light to react for 3 hours, followed by concentration under reduced pressure. Finally, the sample was freeze-dried and then stored at room temperature.

### Example 2

### Structural Analysis of the Thiolated Chitosan

2.1 Modification (Grafting) Efficiency

2.1.1 Ninhydrin Assay

The thiolated chitosan sample (after modification) prepared in Example 1 and the unmodified chitosan sample (control group) were subjected to ninhydrin analysis to detect the content of amino groups. The sample to be tested was dissolved in an acetic acid solution (0.01M) with a concentration of 0.5 mg/ml to form the sample solution to be tested. In addition, as to the acetic acid buffer solution (2M, pH 5.2), 19 grams of sodium acetate (CH₃COOH.3H₂O) was weighed and dissolved in 50 mL of deionized water, followed by addition of 3.6 mL glacial acetic acid and dilution with deionized water to obtain a total volume of 100 mL. 3 g of ninhydrin crystals was dissolved in 100 mL of 95% alcohol solution to form a ninhydrin reagent. During the measurement, 0.25 ml of the acetic acid buffer solution and 0.25 ml of the ninhydrin reagent was added into 0.5 ml of the sample solution to be tested, which was centrifuged to collect the supernatant after incubating at 70° C. for 40 minutes. The absorbance was measured at 570 nm and the calibration line was drawn by glycine. Table 1 shows the results of the absorbance value and the content of amino groups calculated based on the calibration curve of glycine.

TABLE 1
Absorbance value results of Ninhydrin assay

| | Chitosan | Thiolated chitosan |
|---|---|---|
| | sample before | sample (after |
| | modification | modification) |
| | (0.5 mg/mL) | (0.5 mg/mL) |
| Absorbance | 0.16 | 0.15 |
| value at 570 nm | | |
| Content of | 7,740 μ mole/g | 7,260 μ mole/g |
| amino groups | | |

According to the number of amino groups calculated in Table 1, the modification rate of the thiolated chitosan sample is about 6.2% ((7,740−7,260)/7,740=6.2%)).

2.1.2 Ellman's Assay

The thiolated chitosan sample (after modification) prepared in Example 1 and the unmodified chitosan sample (control group) were subjected to Ellman's assay for detecting the thiol content. The sample to be tested was dissolved in the acetic acid solution (0.01M) with a concentration of 2 mg/ml to form the sample solution to be tested. In addition, 3 mg of 5,5′-dithiobis(2-nitrobenzoic acid) was taken and dissolved in 10 ml 0.5M phosphate buffer (pH 8) to formulate the Ellman's reagent. During the measurement, 0.25 ml of the phosphate buffer (pH 8) and 0.5 ml of the Ellman's reagent were added into 0.25 ml of the sample solution to be tested and mixed in the dark. The supernatant was collected after incubating at 37° C. for 60 minutes. The absorbance value was measured at 450 nm and the calibration line was drawn by cysteine (Carbohydrate polymers 179 (2018) 42-49). Table 2 shows the results of the absorbance value and the thiol number calculated based on the calibration curve of cvsteine.

TABLE 2
Absorbance value results of Ellman's assay

| | Chitosan | Thiolated chitosan |
|---|---|---|
| | sample before | sample (after |
| | modification | modification) |
| | (2 mg/mL) | (2 mg/mL) |
| Absorbance | 0.043 | 0.388 |
| value at 450 nm | | |
| Thiol number | 0 μ mole/g | 255 μ mole/g |

The above results show that the thiolated chitosan according to the present invention is confirmed to be partially grafted, with a modification rate of about 6.2% and 255 μmole/g.

2.2 Spectroscopy Analysis

2.2.1 Infrared Spectroscopy Analysis

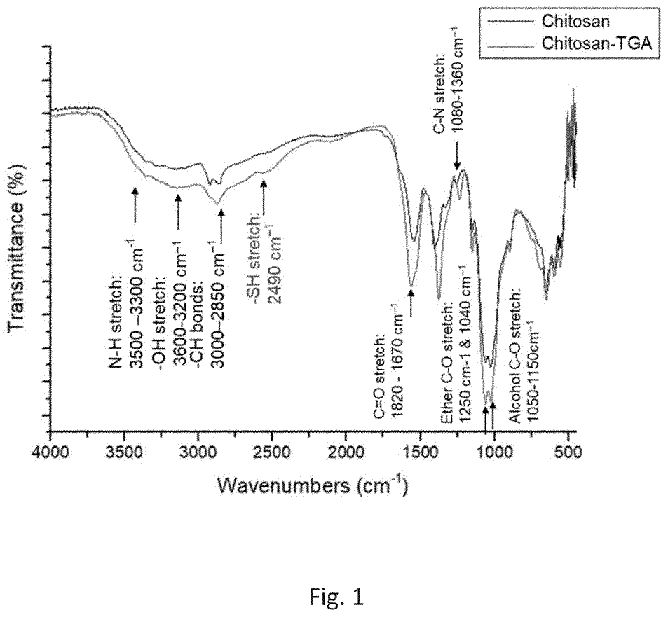

The thiolated chitosan sample (after modification) prepared in Example 1 and the unmodified chitosan sample (control group) were analyzed by infrared spectroscopy to identify the types of functional groups on the material. It adopted the total reflection (ATR) model, the detection wave number ranged from 4000 cm−1 to 450 cm−1, and the number of scans was 16 times. FIG. 1 shows the results.

As shown in FIG. 1, 3600−3200 cm⁻¹ is the absorption peak of —OH swing, 3500−3300 cm⁻¹ is the absorption peak of —NH swing, 2490 cm⁻¹ is the absorption peak of —SH swing, 1820−1670 cm⁻¹ is the absorption peak of C═O swing, 1250 cm⁻¹ & 1040 cm⁻¹ are the absorption peaks of ether C—O swing, and 1050-1150 cm⁻¹ is the absorption peak of alcohol C—O swing. From the Fourier Transform Infrared (FTIR) spectrum, it can be seen that the structure of the chitosan sample and the thiolated chitosan sample are very close, there is only a slight difference, and both have the absorption peaks chitosan should have, such as: 3600−3200 cm⁻¹ being the absorption peak of —OH swing, 3000−2850 cm⁻¹ being the absorption peak of —CH stretching, etc. The only difference lies in that the thiolated chitosan sample additionally has absorption peaks of thiols at 2490 cm⁻¹. Therefore, it can be confirmed that the thiolated chitosan sample has thiol functional groups.

2.2.2 Nuclear Magnetic Resonance (NMR) Spectroscopy Analysis

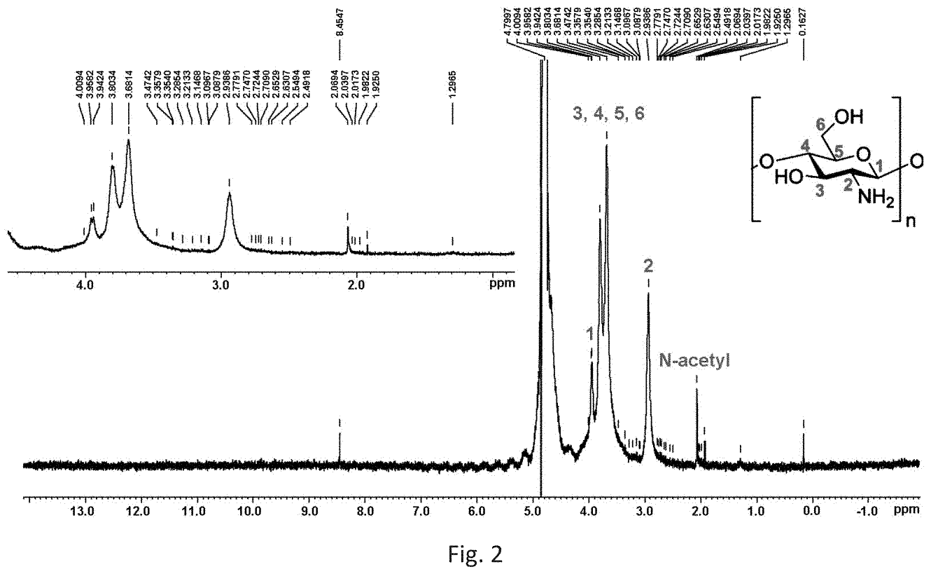

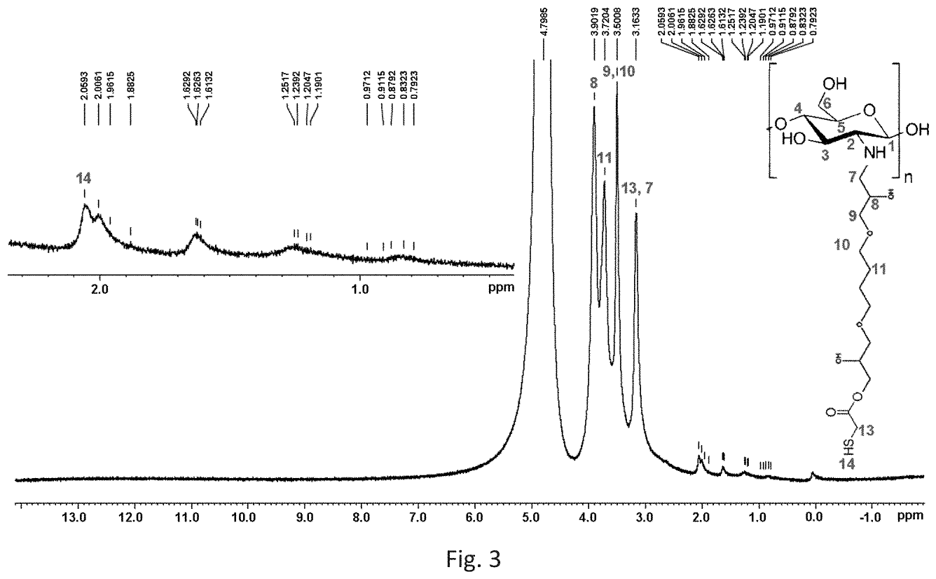

The thiolated chitosan sample (after modification) prepared in Example 1 and the unmodified chitosan sample (control group) were subjected to ¹H-NMR spectroscopy analysis to identify the structure. FIGS. 2 and 3 show the results.

FIG. 2 shows the ¹H-NMR spectrum results of the chitosan sample. The results show that H1 appears at 4.0 ppm, H2 appears at 2.9 ppm, and H3, 4, 5, and 6 are distributed between 3.8 ppm and 3.5 ppm, which are very similar to the results of known chitosan research. It is worth noting that the acetyl peak at 2.0 ppm is significantly higher, indicating a higher degree of deacetylation (International journal of biological macromolecules 94 (2017) 96-105; biosensors 16 (2014) 18; Journal of Materials Chemistry B 1(9) (2013) 1241-1248).

FIG. 3 shows the ¹H-NMR spectrum results of the thiolated chitosan sample. The results shows that the peak of BDDE as the cross-linking agent increases, which is judged that the cross-linking agent did cross-link with chitosan, and the shape of the 2.0 ppm peak changes (the peak of —SH in thioglycolic acid is also here). Although it overlaps with the acetyl group, it can be judged that the test sample is thiolated chitosan in view of the change in the shape of the peak and the results of the above-mentioned infrared spectroscopy analysis and Ellman's assay.

2.3 Biocompatibility Analysis

2.3.1 Water Soluble Tetrazolium Salt-1 (WST-1) Assay

WST-1 is a water-soluble tetrazolium salt reagent that can interact with Nicotinamide Adenine Dinucleotide (NADH) mitochondrial dehydrogenase in mitochondrial electron transport chain in a cell to produce formazan with a darker color. With this feature, cell viability can be assessed by reading the absorbance at 450 nm (Cell viability assays, (2016); Analytical Communications 36(2) (1999) 47-50).

According to ISO10993-1, the cytotoxicity test was carried out. The cells were IEC-6 cell lines and were cultured in Dulbecco's Modified Eagle's Medium (DMEM). The positive control group was a polyurethane (ZDEC) film, and the negative control group was a high-density polyethylene (HDPE) film.

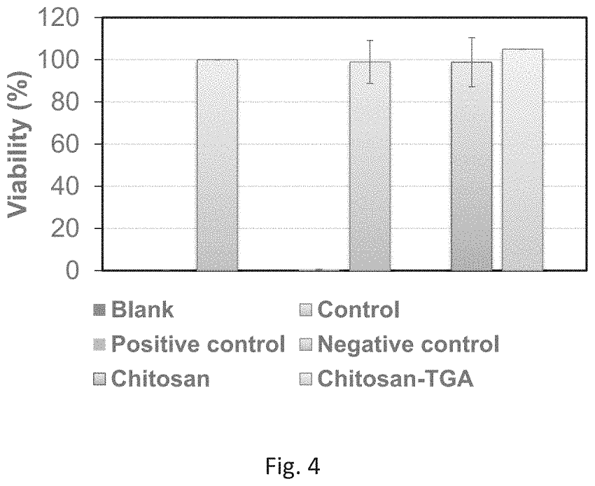

The sample to be tested was dissolved in a 0.01M acetic acid solution to form a sample solution to be tested with a concentration of 3 mg/ml, which was mixed with the culture medium at a volume ratio of 1:5 to form an extract. The cells were planted in a 96-well plate with a cell density of 10,000 cells/well. After culturing for a period of time for attachment, the culture medium was replaced with the extract. After one day of incubation, the extract was removed, washing with phosphate buffered saline (PBS) was carried out for three times, and then the culture medium containing WST-1 was placed in the well plate for 2 hours. The absorbance value at 450 nm was read by enzyme immunoassay ELISA Reader. FIG. 4 shows the results.

The results show that both the thiolated chitosan sample (after modification) and the unmodified chitosan sample (control group) have very good biocompatibility and no obvious cytotoxicity.

2.3.2 Live/Dead Assay

The dyes used were Calcein AM, Propidium iodide (PI), and Hoechst. Calcein AM can pass through the cell membrane and react with esterases to produce strong green fluorescence (Ex/em: 496 nm/515 nm). Because dead cells lack active esterases, they will not emit fluorescence. Although PI cannot cross the cell membrane, it can bind to DNA through the damaged cell membrane, and will emit red fluorescence (Ex/em: 535 nm/617 nm) after binding, which can be used to mark dead cells. Hoechst is also a dye that can penetrate the cell membrane, and emits blue fluorescence (Ex/em: 350 nm/461 nm) after binding to DNA, which can mark the position of the cell nucleus (Cancer research 53(6) (1993) 1332-1337 ; Anti-cancer drugs 6(4) (1995) 522-532).

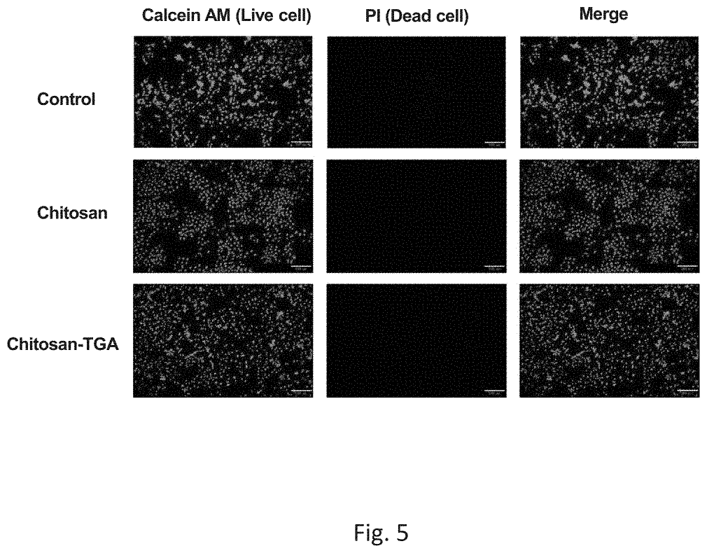

The sample to be tested was dissolved in a 0.01M acetic acid solution to form a sample solution to be tested with a concentration of 3 mg/ml, which was then mixed with the culture medium at a volume ratio of 1:5 to form an extract. The cells were planted in a 24-well plate with a cell density of 10,000 cells/well. After culturing for a period of time for attachment, the culture medium was replaced with the extract. After one day or three days of incubation, the extract was removed, washing with phosphate buffered saline (PBS) was carried out for three times, and then the PBS containing calcein AM (5 μl/ml), propidium iodide (1 μl/ml) and Hoechst (1 μl/ml) was added for staining for 15 minutes. Washing with PBS was carried out for five times, and the degree of apoptosis was observed through a fluorescence microscope to evaluate the biocompatibility. FIG. 5 shows the results.

The results show that the cells did not show obvious apoptosis, corresponding to the results of WST-1. The thiolated chitosan sample (after modification) and the unmodified chitosan sample (control group) have no obvious toxicity to the cells, showing excellent biocompatibility.

2.4 Analysis of the Attachment Ability Using the In Vitro Gastrointestinal Tract Simulation Model

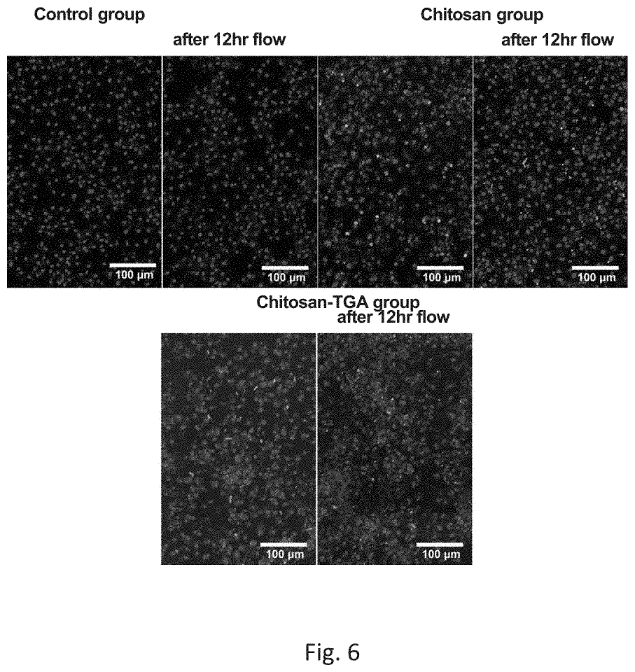

With reference to the design of Hyun Jung Kim et al. (2012), the adhesion ability of the material to mucosa was tested by in vitro intestinal model (Lab on a Chip 12(12) (2012) 2165-2174). The micro-channel device (Germany ibidi company, model μ-Slide I 0.8 Luer) had a channel length of 50 mm, a cross-sectional area of 4 mm² (a width of 5 mm and a height of 0.8 mm), a bottom area of 2.5 cm², and a total capacity of 200 μl. First, the small intestinal mucosal cells IEC-6 were planted into the flow channel at a cell concentration of 2.9×10⁶/ml. After being attached, the culture medium containing Did dye (2.5 μl/ml) and Hoechst (1 μl/ml) was added. After 15 minutes, fresh culture medium was used to change medium, and then the culture medium containing the material to be tested was added at a concentration of 3 mg/ml. The material was allowed to adhere to the mucosa for 40 minutes to simulate the condition that the material enters the duodenum and stays there for 40 minutes. After completion, the material was observed with a fluorescent microscope and then impacted for 12 hours using the culture medium at a flow rate of 750 μl/hr. The attachment of the material after the human body ate was observed. FIG. 6 shows the results.

The results show that in the unmodified chitosan sample group (control group), there was no significant decrease in the number of cells before and after scouring, indicating that the flow rate in this experiment did not cause too strong shear stress on the cells. In the unmodified chitosan sample group (control group), it could be observed that although there were chitosan signals before and after scouring, after 12 hours of scouring, the chitosan appeared agglomerated and the signal of chitosan on the cell surface had also been greatly reduced. In contrast, in the thiolated chitosan sample group (after modification), it could be observed that the signal of the material was very obvious before and after scouring, and there was no significant loss of signal after scouring. Therefore, the thiolated chitosan did exhibit better adhesion properties than the unmodified chitosan.

2.5 Obese Animal Model

This experiment was to test the effect of the thiolated chitosan (after modification) on body weight control in obese rats. The experimental animals were Wistar rats. After the animals were purchased, groups with similar body weights were selected for the experiment, and they were divided into the following groups: (1) Control, (2) 1-fold the unmodified chitosan group (lx Chitosan), (3) 1-fold the thiolated chitosan group (1×Chitosan-TGA), and (4) 0.5-fold the thiolated chitosan group (0.5×Chitosan-TGA). Each group had unlimited feed supply. There were three orifice feeding times per week (Monday, Wednesday, and Friday). After six weeks, the metabolic cage was used to collect urine, blood collection and sacrifice were carried out through the heart, and the body was then opened to take out the organs for pathological tissue sections, as well as blood biochemical analysis and urinalysis. The control group was fed saline, and the other three groups were fed test materials (1-fold the unmodified chitosan group, 1-fold the thiolated chitosan group, 0.5-fold the thiolated chitosan group). The dosage was based on the estimation method announced by the US Food and Drug Administration (FDA), taking 6.2 times the recommended daily intake per kilogram of body weight (/kg bw/day) of the human body as 1-fold the dose for rats. Therefore, in this experiment, rats were given 1-fold dose, being 0.31 g of test material (chitosan or thiolated chitosan) per kilogram of body weight per day, and 0.5-fold dose, being 0.16 g of test material (chitosan or thiolated chitosan) per kilogram of body weight per day.

2.5.1 Animal Body Weight Analysis

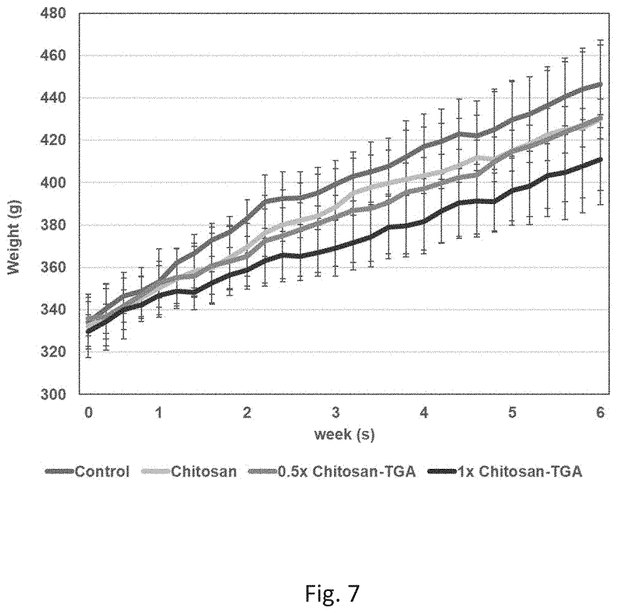

FIG. 7 is a graph showing the daily body weight of animals in each group. The results show that the initial average body weight of each group of animals was about 330 g. As time progressed, the weight of the control group increases the most. It is speculated that the oral feeding technique has no substantial effect on body weight. Compared with the control group, the other three groups show a trend of slowing weight gain. In particular, 1-fold the thiolated chitosan group has a significant effect on inhibiting weight gain, which is better than 1-fold the chitosan group and 0.5-fold the thiolated chitosan group at the same concentration. Furthermore, the longer the time, the more obvious the difference.

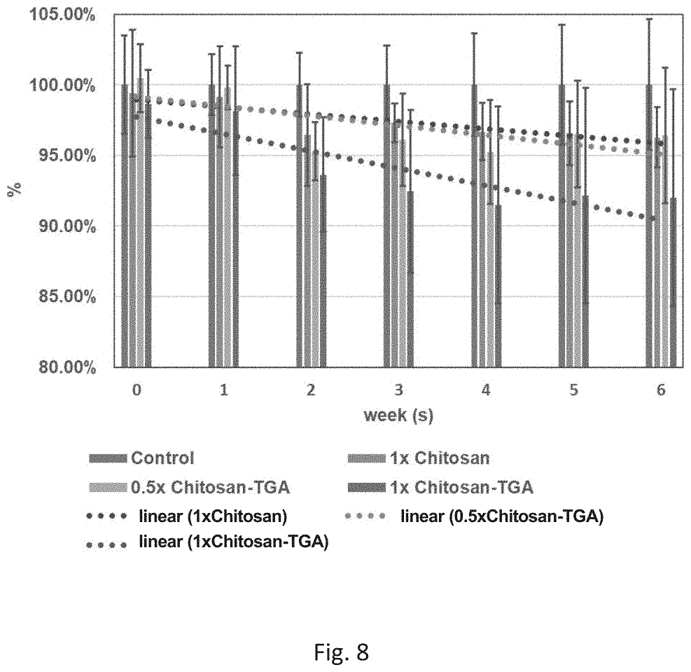

FIG. 8 shows the weekly weight percentages of the animals in three groups, including 1-fold the unmodified chitosan group, 1-fold the thiolated chitosan group, and 0.5-fold the thiolated chitosan group, which were calculated based on the average weight of the animals in the control group as 100% of the current week. The results show that the body weights of the three groups of animals in each week were lower than those of the control group. In particular, the weight difference between 1-fold the thiolated chitosan group and Control was the largest, which was significantly greater than the weight difference of 1-fold the chitosan group and the control group at the same concentration and the weight difference between 0.5-fold the thiolated chitosan group and Control, and the longer the time, the more obvious the difference.

The above results show that the thiolated chitosan is effective in reducing animal weight gain, which is better than the unmodified chitosan.

2.5.2 Animal Tissue Analysis

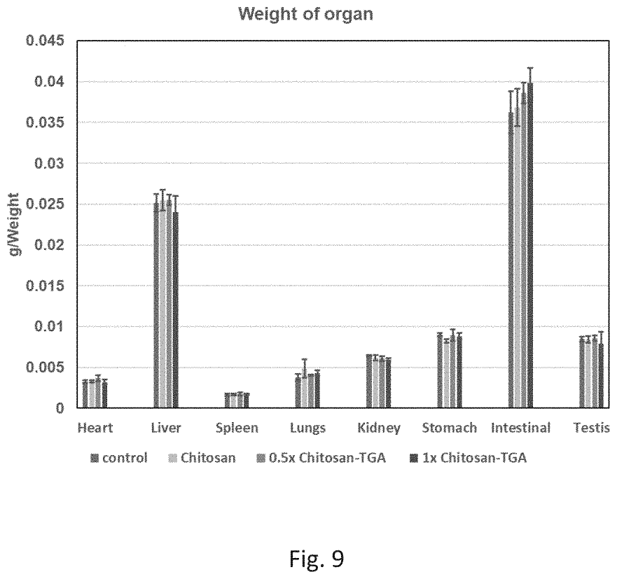

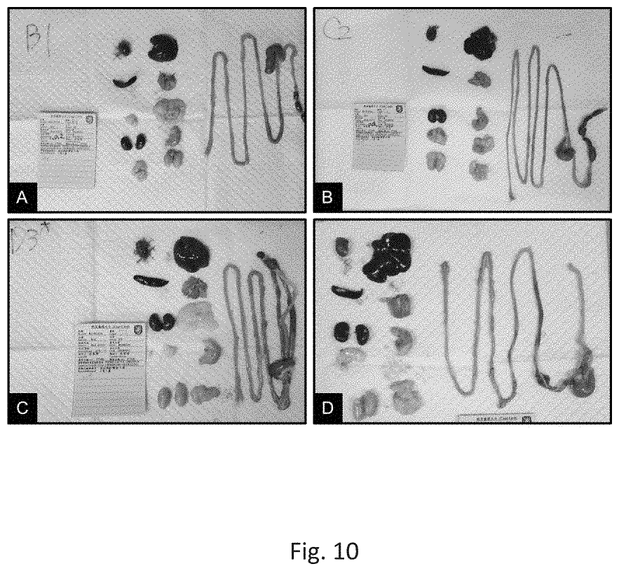

After the animals were sacrificed, the organs were taken out, weighed, and immediately fixed with formalin for 2 days before proceeding with tissue sectioning. FIG. 9 shows the results of organ weight analysis of animals in each group. FIG. 10 shows photos of the appearance of the organs of animals in each group. Previous studies have shown that the increase in organ weight is related to organ inflammation (Archives of surgery 123(12) (1988) 1519-1524). The results show that there was no significant weight difference in the organs of the animals in each group, and there was no significant swelling. This means that the tested chitosan does not cause adverse inflammatory reactions in the animal body, and it is mutually verified with the results of the above cell test that both the thiolated chitosan sample (after modification) and the unmodified chitosan sample (control group) have good biocompatibility.

2.5.3 Serum Chemistry Analysis

Table 3 shows the serum biochemical analysis values.

| | | unmodified | 1-fold the thiolated |
|---|---|---|---|
| | Control | Chitosan | chitosan group |
| GOT(U/L) | 86.3 | 108.7 | 98.0 |
| GPT(U/L) | 32.0 | 40.0 | 32.7 |
| GLU(mg/dl) | 157.7 | 181.3 | 150.7 |
| BUN(mg/dl) | 14.7 | 14.4 | 14.2 |
| CRE(mg/dl) | 0.2 | 0.3 | 0.2 |
| UA(mg/dl) | 1.2 | 1.6 | 1.4 |
| TCHO (mg/dl) | 81.7 | 64.7 | 61.3 |
| HDL-C (mg/dl) | 68.3 | 27.3 | 55.7 |
| TG (mg/dl) | 69.0 | 62.3 | 62.7 |

GOT: aspartate transaminase
GPT: glutamic-pyruvic transaminase
GLU: gluclose
BUN: blood urea nitrogen
CRE: creatinine
UA: uric acid
TCHO: Total Cholesterol
HDL-C: high density lipoprotein-cholesterol
TG: triglyceride

The results show that the values of the unmodified chitosan group and the thiolated chitosan group are within the safety range of literature reference, indicating that the unmodified chitosan and the thiolated chitosan are not toxic to the liver and kidney. In addition, the blood lipid related indicators, such as the total cholesterol (TC) and triglycerides (TG), in the unmodified chitosan group and the thiolated chitosan group were lower than those of the control group, indicating that it has the function of reducing blood lipids and cholesterol.

2.5.4 Hematology & Urinalysis

Table 4 shows the results of blood analysis. The results show that the blood analysis items of each group fell within the normal range, indicating that neither the unmodified chitosan nor the thiolated chitosan would trigger a severe immune response in the animals. Table 4 shows the results of urinalysis. The results show that the urinalysis items of each group fell within the normal range, indicating that neither the unmodified chitosan nor the thiolated chitosan would cause damage to the animal's kidney function. Overall, both the thiolated chitosan sample (after modification) and the unmodified chitosan sample (control group) have good biocompatibility.

TABLE 4
Blood analysis results

| | | unmodified | 1-fold the thiolated |
|---|---|---|---|
| | Control | Chitosan | chitosan group |
| WBC (10³/μl) | 11.46 | 12.87 | 14.74 |
| NE (10³/μl) | 1.94 | 2.90 | 3.49 |
| LY (10³/μl) | 2.56 | 4.53 | 4.11 |
| MO (10³/μl) | 0.26 | 0.41 | 0.30 |
| EO (10³/μl) | 0.03 | 0.03 | 0.13 |

WBC: white blood cell
NE: neutrophils
LY: lymphocyte
MO: monocytes
EO: eosinophils

TABLE 5
Urinalysis results

| | | unmodified | 1-fold the thiolated |
|---|---|---|---|
| | Control | Chitosan | chitosan group |
| GLU | Negative | Negative | Negative |
| BIL | Negative | Negative | Negative |
| KET | Negative | Negative | Negative |
| SG | 1.0 | 1.0 | 1.0 |
| BLO | Negative | Negative | Negative |
| PH | 6.8 | 7.1 | 7.0 |
| PRO(mg/dL) | Negative | Negative | Negative |
| URO(E.U./dL) | 0.2 | 0.2 | 0.2 |
| NIT | Negative | Negative | Negative |
| LEU | Negative | Negative | Negative |

GLU: glucose
BIL: Bilirubin
KET: Ketone
SG: specific gravity
BLO: Occult blood
PH: PH
PRO: protein
URO: urobilinogen
NIT: nitrate
LEU: Leukocyte

### 3. Conclusion

This study provides a thiolated chitosan material, which can effectively reduce the body weight gain of animals and can be used for body weight control. The thiolated chitosan shows no cytotoxicity in cell tests and good adhesion to mucous membranes, and in animal tests, it has been shown that oral administration can effectively reduce the body weight gain of animals and have the effect of lowering blood lipids and cholesterol without damages to organs and tissues as well as adverse reactions such as inflammation. The blood urinalysis is normal with good biocompatibility.

The EndoBarrier device developed in the prior art is delivered to the middle of the stomach and the duodenum through a gastroscope in an invasive manner, and a plastic film is used to block the intestinal absorption of nutrients to achieve the purpose of body weight control. However, invasive delivery causes pain and risks to the patient. Gastrointestinal peristalsis may cause the device to fall off, and the device may cause oppression to the surrounding tissues, resulting in abnormal proliferation damage. Long-term blocking of nutrient absorption will cause harm to the patient's health, and the device must be taken out within a certain period of time.

In contrast, the thiolated chitosan material provided in this study overcomes the shortcomings of the prior art. It is orally delivered and expected that, after entering the front of the small intestine, it will adhere to the intestinal mucosa to form a film barrier for avoiding direct contact between food and the intestinal mucosa, thereby reducing absorption and achieving the effect of body weight control. This method avoids invasive delivery, and the material is safe, non-toxic and degradable. It does not require a second operation to remove the device out, and provides a simple, convenient, low-risk and gentle and effective method to achieve the purpose of body weight control or treatment of overweight or obesity.

What is claimed is:

1. A method for body weight control in a subject in need thereof, comprising administering an effective amount of an a thiolated chitosan to the subject.

2. The method of claim 1, wherein the thiolated chitosan comprises a plurality of thiol groups covalently bonded to a chitosan moiety thereof.

3. The method of claim 1, wherein the thiolated chitosan is partially thiolated.

4. The method of claim 1, wherein the thiolated chitosan has a degree of thiolation of 0.5% to 50%.

5. The method of claim 1, wherein the thiolated chitosan has a degree of thiolation of 30 to 5,000 μmol/g.

6. The method of claim 1, wherein the thiolated chitosan has an average molecular weight of 5 to 2,000 kDa.

7. The method of claim 1, wherein the thiolated chitosan is represented by formula I: 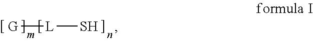
wherein 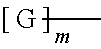
represents a chitosan moiety, G is a monomer unit of the chitosan moiety, including a N-acetyl-D-glucosamine monomer and a glucosamine monomer, in which the monomer units are connected to form the chitosan moiety, and m represents a total number of the monomers, being an integer from 30 to 12,500;
SH represents a thiol group, and L represents a linker molecule moiety; and
n is an integer from 1 to 6,250 and n is not more than m.

8. The method of claim 7, wherein L is a bond, or a molecular moiety including one or more functional groups containing heteroatoms, which is selected from the group consisting of amido, amino, carbonyl, carbamate, urea, disulphide, ether, succinyl, succinamidyl and a combination thereof.

9. The method of claim 7, wherein L is —R—C(O)—R′—, where R and R′ are each independently a bond, or R and R′ are each independently a C1-20 alkylene group, optionally containing 1 to 3 oxygen atoms in its main chain, unsubstituted or substituted with 1 to 3 hydroxyl groups (—OH).

10. The method of claim 7, wherein L is —R—C(O)—R′—, where R is a C1-6 alkylene group, and R′ is a C1-18 alkylene group containing 1 to 3 oxygen atoms in its main chain and substituted with 1 to 3 hydroxyl groups (—OH).

11. The method of claim 7, wherein L is represented by formula Ia

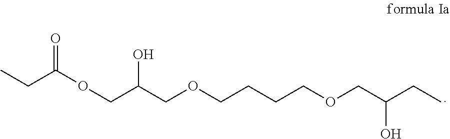 [SMILES: CCC(=O)OCC(O)COCCCCOCC(O)CC]

12. The method of claim 1, wherein m is an integer from 30 to 300, an integer from 300 to 1,000, an integer from 1,000 to 2,500, or an integer from 2,500 to 10,000.

13. The method of claim 1, wherein the thiolated chitosan has an average molecular weight of 5 to 50 kDa, 50 to 200 kDa, 200 to 500 kDa, or 500 to 2,000 kDa.

14. The method of claim 1, wherein the thiolated chitosan is administered in an amount effective in slowing down body weight gain of the subject.

15. The method of claim 1, wherein the thiolated chitosan is administered in an amount effective in treating or avoiding overweight or obesity of the subject.

16. The method of claim 1, wherein the thiolated chitosan is formulated with a physiologically acceptable carrier to form a composition

17. The method of claim 16, wherein the composition is in a form of food or medicine.

18. The method of claim 16, wherein the composition is in a form of tablets, capsules, powder, emulsions and aqueous suspensions.

19. The method of claim 1, wherein the thiolated chitosan is administered orally.

20. The method of claim 19, wherein the thiolated chitosan is administered daily or every other day for a period of 1 to 6 weeks or longer.
